(19) United States
(12) Patent Application Publication
(10) Pub. No.: US 2007/0209661 A1
(43) Pub. Date: Sep. 13, 2007

(54) Dry powder inhaler with aeroelastic dispersion mechanism

(76) Inventors: HUGH SMYTH, ALBUQERQUE, NM (US); CHARLES RANDALL TRUMAN, ALBUQUERQUE, NM (US)

Correspondence Address: Alberto A. Leon, Esquire, PO Box 30684, Albuquerque, NM, 87190, US

(21) Appl. No.: 11/713,180

(22) Filed: Mar. 2, 2007

Related U.S. Application Data
(60) Provisional application No. 60/778,878, filed on Mar. 3, 2006.

(51) Int. Cl. A61M 15/00; A61M 16/10

(52) U.S. Cl. 128/203.12; 128/203.15

(57) ABSTRACT

The present invention comprises a dry powder inhaler (DPI) that uses a patient's inhalation flow to concentrate energy in an aeroelastic element for deaggregation and dispersion of a powder dose. The result is a DPI that delivers a dose independent of inspiratory abilities of the patient, solving a major problem of conventional DPIs. Increased tension on the aeroelastic element causes higher frequency vibrations and improved powder dispersion. The tension of the aeroelastic element can be modified prior to dispensing the DPI to the patient, allowing for individualization for single patients or groups of patients. In addition, the DPI has features that increase the turbulence of the airflow as it passes through the device, further increasing the dispersion and deaggregation of the powder. The DPI can hold a single dose or multiple doses. The powder doses can be dispensed directly onto the aeroelastic element, or may be in adjacent blister packaging.

### CROSS-REFERENCE TO RELATED APPLICATION

I hereby claim the benefit under Title 35, United States Code Section 119(e) of any United States Provisional Application(s) listed below:
Application No. 60/778,878
Filing Date: Mar. 3, 2006

### TECHNICAL FIELD OF THE INVENTION

The present invention relates generally to inhalers, dry powder inhalers, inhalation flows and more specifically to a method of using dry powder inhalers.

### BACKGROUND OF THE INVENTION

Dry powder inhalers (“DPIs”) represent a promising alternative to pressurized meted dose inhaler (“pMDI”) devices for delivering drug aerosols without using CFC propellants. See generally, Crowder et al., 2001: an Odyssey in Inhaler Formulation and Design, Pharmaceutical Technology, pp. 99-113, July 2001; and Peart et al., New Developments in Dry Powder Inhaler Technology, American Pharmaceutical Review, Vol. 4, n. 3, pp. 37-45 (2001). Martonen et al. 2005 Respiratory Care, Smyth and Hickey American Journal of Drug Delivery, 2005

Typically, the DPIs are configured to deliver a powdered drug or drug mixture that includes an excipient and/or other ingredients. Conventionally, many DPIs have operated passively, relying on the inspiratory effort of the patient to dispense the drug provided by the powder. Unfortunately, this passive operation can lead to poor dosing uniformity since inspiratory capabilities can vary from patient to patient, and sometimes even use-to-use by the same patient, particularly if the patient is undergoing an asthmatic attack or respiratory-type ailment which tends to close the airway.

Generally described, known single and multiple dose DPI devices use: (a) individual pre-measured doses, such as capsules containing the drug, which can be inserted into the device prior to dispensing; or (b) bulk powder reservoirs which are configured to administer successive quantities of the drug to the patient via a dispensing chamber which dispenses the proper dose. See generally Prime et al., Review of Dry Powder Inhalers, 26 Adv. Drug Delivery Rev., pp. 51-58 (1997); and Hickey et al., A new millennium for inhaler technology, 21 Pharm. Tech., n. 6, pp. 116-125 (1997).

In operation, DPI devices desire to administer a uniform aerosol dispersion amount in a desired physical form (such as a particulate size) of the dry powder into a patient's airway and direct it to a desired deposit site. If the patient is unable to provide sufficient respiratory effort, the extent of drug penetration, especially to the lower portion of the airway, may be impeded. This may result in premature deposit of the powder in the patient's mouth or throat.

A number of obstacles can undesirably impact the performance of the DPI. For example, the small size of the inhalable particles in the dry powder drug mixture can subject them to forces of agglomeration and/or cohesion (i.e., certain types of dry powders are susceptible to agglomeration, which is typically caused by particles of the drug adhering together), which can result in poor flow and non-uniform dispersion. In addition, as noted above, many dry powder formulations employ larger excipient particles to promote flow properties of the drug. However, separation of the drug from the excipient, as well as the presence of agglomeration, can require additional inspiratory effort, which, again, can impact the stable dispersion of the powder within the air stream of the patient. Unstable dispersions may inhibit the drug from reaching its preferred deposit/destination site and can prematurely deposit undue amounts of the drug elsewhere.

Further, many dry powder inhalers can retain a significant amount of the drug within the device, which can be especially problematic over time. Typically, this problem requires that the device be disassembled and cleansed to assure that it is in proper working order. In addition, the hygroscopic nature of many of these dry powder drugs may also require that the device be cleansed and dried periodically.

A number of different inhalation devices have been designed to attempt to resolve problems attendant with conventional passive inhalers. For example, U.S. Pat. No. 5,655,523 discloses and claims a dry powder inhalation device which has a deagglormeration-aerosolization plunger rod or biased hammer and solenoid: U.S. Pat. No. 3,948,264 discloses the use of a battery-powered solenoid buzzer to vibrate the capsule to effectuate the efficient release of the powder contained therein. Those devices are based on the proposition that the release of the dry powder can be effectively facilitated by the use of energy input independent of patient respiratory effort.

U.S. Pat. No. 6,029,663 to Eisele et al. discloses and claims a dry powder inhaler delivery system with carrier disk capable of rotating, having a blister shell sealed by a shear layer that uses an actuator that tears away the shear layer to release the powder drug contents. The device also includes a hanging mouthpiece cover that is attached to a bottom portion of the inhaler.

U.S. Pat. No. 5,533,502 to Piper discloses and claims a powder inhaler using patient inspiratory efforts for generating a respirable aerosol. The Piper invention also includes a cartridge capable of rotating, holding the depressed wells or blisters defining the medicament holding receptacles. A spring-loaded carriage compresses the blister against conduits with sharp edges that puncture the blister to release the medication that is then entrained in air drawn in from the air inlet conduit so that aerosolized medication is emitted from the aerosol outlet conduit.

Crowder et al. describe a dry powder inhaler in U.S. Pat. No. 6,889,690 comprising a piezoelectric polymer packaging in which the powder for aerosolization is simulated using non-linear signals determined a priori for specific powders.

In recent years, dry powder inhalers (DPIs) have gained widespread use, particularly in the United States. Currently, the DPI market is estimated to be worth in excess of US $4 billion. Dry powder inhalers have the added advantages of a wide range of doses that can be delivered, excellent stability of drugs in powder form (no refrigeration), ease of maintaining sterility, non-ozone depletion, and they require no press-and-breathe coordination.

There is great potential for delivering a number of therapeutic compounds via the lungs (see for example Martonen T., Smyth H D C, Isaccs K., Burton R., “Issues in Drug Delivery: Dry Powder Inhaler Performance and Lung Deposition”: Respiratory Care. 2005, 50(9); and Smyth H D C., Hickey, A J., “Carriers in Drug Powder Delivery: Implications for Inhalation System Design”: American Journal of Drug Delivery, 2005, 3(2), 117-132). In the search for non-invasive delivery of biologics (which currently must be injected), it was realized that the large highly absorptive surface area of the lung with low metabolic drug degradation, could be used for systemic delivery of proteins such as insulin. The administration of small molecular weight drugs previously administered by injection is currently under investigation via the inhalation route either to provide non-invasive rapid onset of action, or to improve the therapeutic ratio for drugs acting in the lung (e.g. lung cancer).

Gene therapy of pulmonary disease is still in its infancy but could provide valuable solutions to currently unmet medical needs. The recognition that the airways may provide a real opportunity for delivering biotech therapeutics in a non-invasive way was recently achieved with Exubera™, an inhaled insulin product. This product has obtained a recommendation for approval by US Food and Drug Administration and will lead to expanded opportunities for other biologics to be administered via the airways.

Key to all inhalation dosage forms is the need to maximize the “respirable dose” (particles with aerodynamic diameters<5.0 μm that deposit in the lung) of a therapeutic agent. However, both propellant-based inhalers and current DPI systems only achieve lung deposition efficiencies of less than 20% of the delivered dose. The primary reason why powder systems have limited efficiency is the difficult balancing of particle size (particles under 5 μm diameter) and strong inter-particulate forces that prevent deaggregation of powders (strong cohesive forces begin to dominate at particle sizes<10 μm) (Smyth H D C., Hickey, A J., “Carriers in Drug Powder Delivery: Implications for Inhalation System Design”: American Journal of Drug Delivery, 2005, 3(2), 117-132). Thus, DPIs require considerable inspiratory effort to draw the powder formulation from the device to generate aerosols for efficient lung deposition (see FIG. 1 for an illustration of typical mechanism of powder dispersion for DPIs). Many patients, particularly asthmatic patients, children, and elderly patients, which are important patient groups for respiratory disease, are not capable of such effort. In most DPIs, approximately 60 L/min of airflow is required to effectively deaggregate the fine cohesive powder. All currently available DPIs suffer from this potential drawback.

Multiple studies have shown that the dose emitted from dry powder inhalers (DPI) is dependent on air flow rates (see Martonen T., Smyth H D C, Isaccs K., Burton R., “Issues in Drug Delivery: Dry Powder Inhaler Performance and Lung Deposition”: Respiratory Care. 2005, 50(9)). Increasing air-flow increases drug dispersion due to increases in drag forces of the fluid acting on the particle located in the flow. The Turbuhaler® device (a common DPI), is not suitable for children because of the low flow achieved by this patient group (see Martonen T., Smyth H D C, Isaccs K., Burton R., “Issues in Drug Delivery: Dry Powder Inhaler Performance and Lung Deposition”: Respiratory Care. 2005, 50(9)).

Considerable intra-patient variability of inhalation rates has been found when patients inhale through two leading DPI devices. That inherent variability has prompted several companies to evaluate ways of providing energy in the inhaler (i.e. “active” DPIs). Currently, there is no active DPI commercially available. The active inhalers under investigation include technologies that use compressed air, piezoelectric actuators, and electric motors. The designs of those inhalers are very complex and utilize many moving parts and components. The complexity of those devices presents several major drawbacks including high cost, component failure risk, complex manufacturing procedures, expensive quality control, and difficulty in meeting specifications for regulatory approval and release (Food and Drug Administration).

Alternatively, powder technology provides potential solutions for flow rate dependence of DPIs. For example, hollow porous microparticles having a geometric size of 5-30 μm, but aerodynamic sizes of 1-5 μm require less power for dispersion than small particles of the same mass. This may lead to flow independent drug dispersion but is likely to be limited to a few types of drugs with relevant physicochemical properties.

Thus there are several problems associated with current dry powder inhaler systems including the most problematic issue: the dose a patient receives is highly dependent on the flow rate the patient can draw through the passive-dispersion device. Several patents describing potential solutions to this problem employ an external energy source to assist in the dispersion of powders and remove this dosing dependence on patient inhalation characteristics. Only one of these devices has made it to market or been approved by regulatory agencies such as the US Food and Drug Administration. Even upon approval, it is likely that these complex devices will have significant costs of manufacture and quality control, which could have a significant impact on the costs of drugs to patients.

The present invention comprises a dry powder inhaler and associated single or multi-dose packaging which holds the compound to be delivered for inhalation as a dry powder. The dry powder inhaler bridges the gap between passive devices and active devices and solves the major issues of each device. The inhaler is a passive device that operates using the energy generated by the patient inspiratory flow inhalation maneuver. However, the energy generated by airflow within the device is focused on the powder by using oscillations induced by airflow across an elastic element. In this way the inhaler can be “tuned” to disperse the powder most efficiently by adjusting the resonance frequencies of the elastic element to match the physicochemical properties of the powder. In addition, the airflow rate required to generate the appropriate oscillations within the device are minimized because some of the energy used to create the vibrations in the elastic element are pre-stored in the element in the form of elastic tension (potential energy). Inhaler performance may be tailored to the lung function of individual patients by modulating the elastic tension. Thus, even patients with poor lung function and those who have minimal capacity to generate airflow during inspiration will able to attain the flow rate required to induce oscillations in the elastic element.

### SUMMARY OF THE INVENTION

This application discloses and claims a highly efficient and reproducible dry powder inhaler which has been developed from a simple design, and which utilizes the patients' inhalation flow to concentrate energy for deaggregation and dispersion of the particles in the aerosol via aeroelastic vibrations. The principles underlying the present invention allows inhaler performance to be significantly improved in terms of efficiency. Further the device and method of the present invention eliminate the inhaler performance's dependence on the inspiratory flow rate of individual patients. The physical principles behind the aeroelastic dispersion mechanism facilitate a simple and low cost inhaler design. Furthermore, inhaler performance may be tailored to the lung function of the patient for optimal individualized drug delivery.

When an elastic structure is subjected to aerodynamic loads its deformations may give rise to new aerodynamic loads, and a fluid-structure interaction results. That interaction may result in several aeroelastic phenomena such as flutter and divergence (See FIGS. 1-2). Typically aeroelasticity is deemed a detrimental phenomenon in the design of airplane wings, bridges, turbines etc. In engineering aeroelastic models, the aerodynamic loads are usually computed by semi-empirical models. The increasing capabilities of modern computers have recently made possible the numerical simulation of fully three-dimensional viscous flows using Computational Fluid Dynamics (CFD) for some realistic engineering problems. Flutter occurs when the fluid surrounding a structure feeds back dynamic energy into the structure instead of absorbing it. Typically a structure will be stable up to a limiting velocity (the flutter velocity) for given conditions then rapidly, even catastrophically, undertake significant dynamic motion. The present invention utilizes aeroelasticity to accomplish the increased dispersion of particles located on or adhered to a thin film within a moving air flow. In addition, predictable amounts of particles can be dispersed even at variable input flow rates. Even further, aeroacoustic emissions resulting from flutter and aeroelastic vibrations may be used in inhaler design to provide positive feedback to the patient indicating that appropriate inhalation flow rates have been achieved, i.e. a whistle or buzz sounds when the minimum effective flow rate is generated.

Material properties and film tension will determine the velocity at which aeroelastic motion or flutter will occur, thus dispersing particles into the moving stream for patient inhalation. Among properties that may be varied are the film stiffness and the tension that is placed on it, polymer film thickness and width, and the length of the film between supports.

Based on the propositions expounded above, it is necessary to modify the flow-field to attain precise drug delivery under the wide range of patient air flow rates. FIG. 2 shows a configuration to create vortex-induced vibration in the film with flow over a bluff body. Periodic forcing by the alternating vortices in the wake of the rod (shown with triangular cross-section) will generate vibration and aeroelastic response in the film. Different sized triangular cross-sections may be inserted to vary the shedding frequency depending on patient flow rate. Film tension may be varied as well.

In a related concept, cavity resonance will acoustically excite the film. The frequency of the acoustic forcing may be varied by changing the geometry of the cavity. The flow separates at the lip of the cavity and impinges near the rear. The depth or the length of the cavity could easily be adjustable within a single device to modify the acoustic forcing frequency to induce aeroelastic response in the film. That configuration may be appropriate if the patient flow rate is too small to induce the necessary aeroelastic response as in FIG. 2.

Some of the most salient advantages of the present invention are: (1) improved inhaler efficiency; (2) flow rate independence; and (3) individualized drug delivery.

Inhaler efficiency is improved by flow-induced vibrations (aeroelastic vibrations) that provide additional dispersion energy directly to the powder. Vibration amplitude, frequency and acceleration may be matched to the forces of adhesion between the powder particles and the aeroelastic substrate to optimize dispersion

Flow-rate independence will be achieved because the fluid mechanical design of the inhaler can ensure that the critical flow rate to achieve aeroelastic response is low, i.e., vibration energy for powder dispersion will be achievable for all patient lung functions. Increases in inhalation flow rate above this critical value will not be necessary for efficient aerosolization and lung delivery.

Modifications to the inhaler (either preset during manufacture or when the medication is dispensed by the pharmacist) will be easily attainable for different patients. For example, pediatric patients with low flow rates and shallower tidal volume may require high frequency vibrations for optimal drug powder dispersion. Higher frequency vibrations can be obtained by increasing the tension force on the aeroelastic element.

### DESCRIPTION OF THE DRAWINGS

FIG. 1: is the airflow at velocity V passing over an aeroelastic membrane (1) under tension, resulting in flutter or vibration of the aeroelastic membrane (in cross-section). The vibration is represented by vertical arrows, and the airflow is represented by horizontal arrows.

FIG. 2: is a configuration to create vortex-induced vibration in an aeroelastic membrane due to airflow over a triangular-shaped rod (2) (in cross-section). The rod causes opposing vortices as airflow passes over and under the rod.

FIG. 3: is a schematic representation of a cross-sectional view of the inhaler of the invention with representations of the major elements of the invention.

FIG. 4: is a schematic representation of the first and second rollers (10) loaded with the aeroelastic membrane with axles in the center of the rollers (15).

FIG. 5: is representation of the preferred embodiment of the dosing applicator.

FIG. 6: is an alternate embodiment of the dosing applicator.

FIG. 7: is a representation of the aeroelastic membrane and its relation to the base clamps (19), upper clamps (20) and tension rods (5). FIG. 7a represents the action that occurs when the advancement means is activated, wherein the upper clamps and tension rods are lifted from the aeroelastic membrane, allowing it to move freely and bring a powder dose (18) in to the center dispensing region. An arrow (21) shows the direction of membrane travel. FIG. 7b shows the powder dose in the center dispensing region and the upper clamps lowered into their resting position. FIG. 7c depicts the final step wherein the tensioner rods return to their resting position, tensioning the aeroelastic membrane at a pre-determined level of tension.

FIG. 8: is a representation of the dispensing mechanism of an alternative embodiment of the invention, wherein a blister strip (22) comprising a series of individual dosing cup (23) filled with a powder dose replaces the aeroelastic membrane and a tensioned aeroelastic element (1) is immediately adjacent to the blister strip. The large arrows depict the direction of airflow across the blister strip and aeroelastic element. The small vertical arrows depict the vibrational motion of the aeroelastic element.

FIG. 9: is a representation of the dispensing mechanism of an alternative embodiment of the invention, wherein a blister strip with multiple dosing cups (24) for different medicaments replaces the aeroelastic membrane and a tensioned aeroelastic element is immediately adjacent to the blister strip.

FIG. 10: is a representation of the dispensing mechanism of an alternative embodiment of the invention, wherein the aeroelastic element is an aeroelastic and deformable membrane (25) with deformable dosing cups (26) that contain the powder dose. As the membrane is stretched by the tensioning rods, the dosing cup deforms and raises the powder dose to the level of the surrounding membrane, where it is easily dispersed upon inhalation by the patient. The horizontal arrows represent the tensioning of the aeroelastic, deformable membrane.

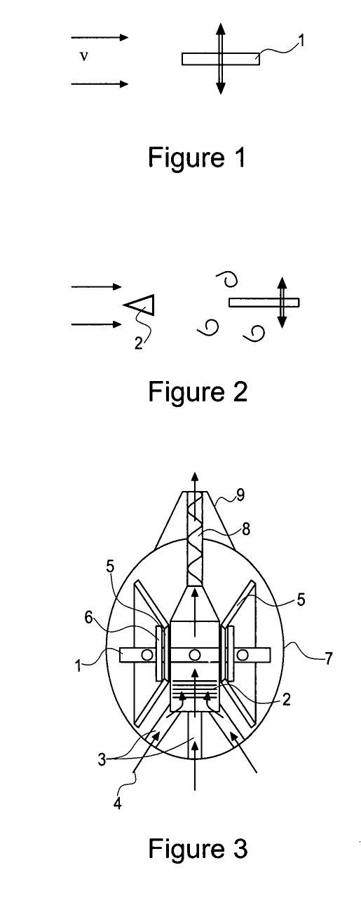

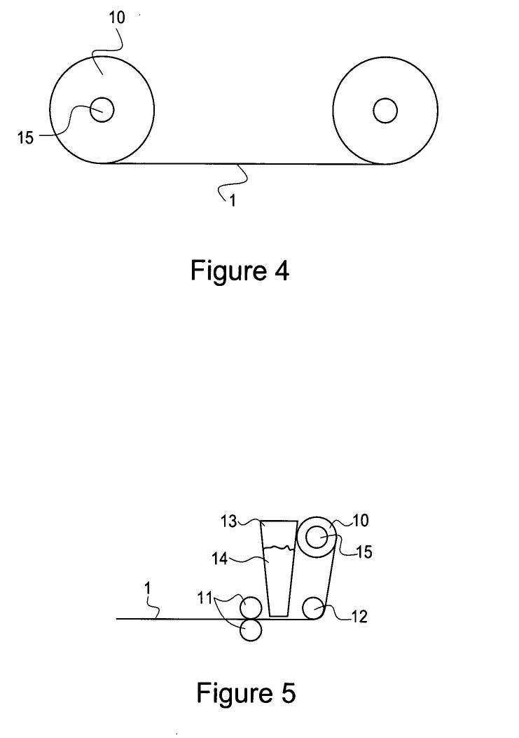

### DETAILED DESCRIPTION OF THE PREFERRED EMBODIMENT OF THE INVENTION

The preferred embodiment of the invention comprises a dry powder inhaler with an integrated assisted dispersion system that is adjustable according to the patients' inspiratory capabilities and the adhesive/cohesive nature of the powder. The inhaler comprises an aeroelastic element that flutters or oscillates in response to airflow through the inhaler. The aeroelastic element provides concentrated energy of the airflow driven by the patient into the powder to be dispersed. The aeroelastic element is preferably a thin elastic membrane held under tension that reaches optimal vibrational response at low flow rates drawn through the inhaler by the patient. The aeroelastic element is preferably adjustable according to the patient's inspiratory capabilities and the adhesive/cohesive forces within the powder for dispersal.

The inhaler itself is a casing with an outer surface (7) and two inner walls that form three distinct chambers inside of the inhaler. The center chamber is essentially open and is the area where air flows through the inhaler upon inhalation by the patient. The center chamber has a front end, which is adjacent to the nozzle (8) and mouthpiece (9), a back end, which is adjacent to the vents or airflow inlets (3), and a center dispensing region, across which the aeroelastic element (1) is stretched.

The inner walls, one right wall and one left wall, form two enclosed chambers, a right chamber to the right of the open center chamber and a left chamber to the left of the open center chamber. Each inner wall has at least one opening, through which the aeroelastic membrane passes. All other elements of the inhaler are found within these enclosed chambers. Two of the elements extend from inside these chambers to the exterior of the inhaler. The first is a dose counter, which indicates to the patient how many doses of medication are remaining in the inhaler. The second is an advancement means, which takes the form of a lever or a dial, which the patient activates to prepare the next dose in the inhaler to be dispensed.

The aeroelastic element engages several elements of the invention. In the preferred embodiment, the aeroelastic element is an elastic membrane with a powder dose, which spans the center dispensing region. The membrane has a used end and an unused end and is wound between two spools, a first spool and a second spool. The first spool holds the unused end, and therefore houses all of the aeroelastic membrane upon installation. The first spool is located in the left chamber and the second spool, which is attached to the used end, is located in the right chamber, resulting in the aeroelastic membrane running through the slot in the left wall across the center dispensing region and through the slot in the left wall onto the second spool. An axle runs through the center of each spool. The axle for the second spool contains a concentric spring, resulting in the aeroelastic membrane being transferred from the first spool to the second spool as the spring-loaded axle is activated by the activating means. Immediately adjacent to the first spool, a roller (12) engages the aeroelastic membrane, resulting in additional tension in the aeroelastic membrane.

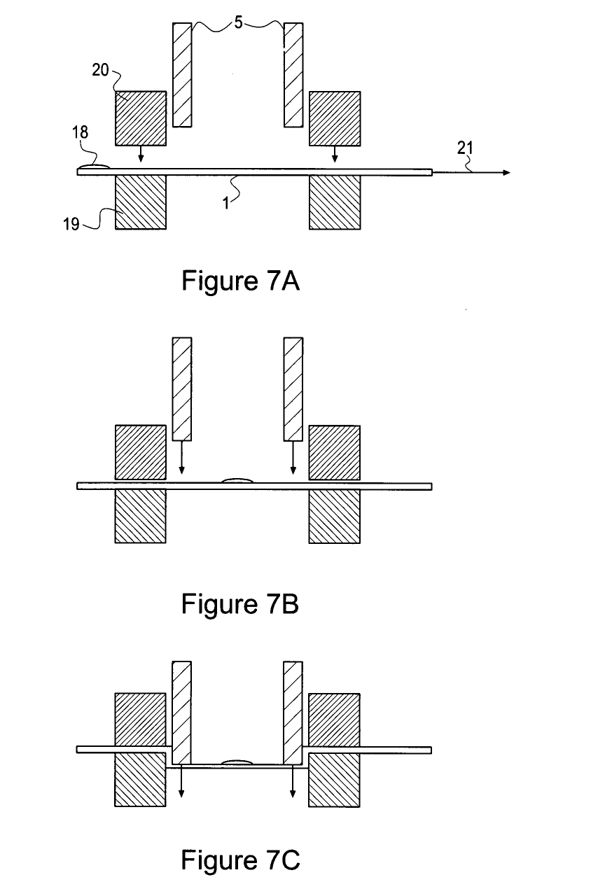

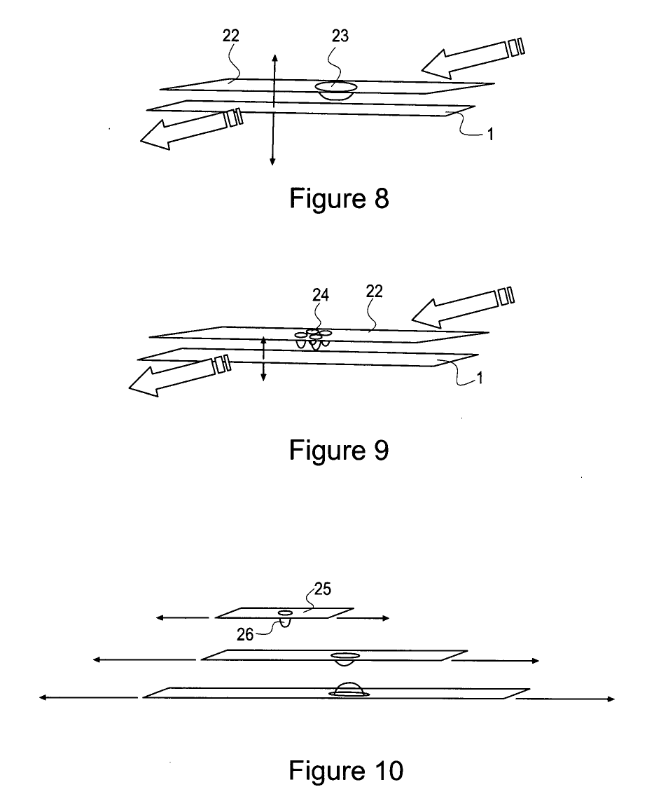

The aeroelastic membrane is held between two pairs of membrane clamps (6). As depicted in FIG. 7, two base clamps (19) are fixedly attached to the floor of the chambers, one in the right chamber and one in the left chamber, upon which the aeroelastic element rests. The clamps are located between the spools and the left and right walls, respectively. Two upper clamps (20) are located above the base clamps. The upper clamps descend atop the base clamps to hold the aeroelastic element in place across the center dispensing region. A crank is movably attached to the two upper clamps. The crank causes the upper clamps to raise from the base clamps when the advancing means is activated and the crank moves. This allows the aeroelastic element to move from the first spool from the second spool and provide the next dose of powder for dispensing to the patient.

Two tensioner rods (21) are located between the upper clamps and the left and right walls and are movably attached to a crank that causes them to descend to a pre-determined level to further tension the aeroelastic element, releasing when the advancing means is activated and the crank moves. The depth to which the tensioner rods descend, and therefore the tension on the aeroelastic element, can be set prior to dispensing the inhaler to the patient, allowing the inhaler to be modified to meet the inspiratory limitations of individual patients or patient groups.

In an alternate embodiment of the invention, tension controllers are attached to the spool axles, allowing the tension of the aeroelastic membrane to be manually fixed prior to the inhaler being dispensed to the patient. The tension is maintained across the spool axles, obviating the need for tension rods.

Certain structural features within the inhaler are included upstream of the aeroelastic element to serve as airflow modifiers to reduce the threshold flow rate at which the aeroelastic element oscillates at the predetermined levels. In the preferred embodiment of the invention, the airflow modifiers are triangular rods (2) extending across the path of the airflow, resulting in vortices as the air passes above and below the triangular rods, as illustrated in FIG. 2.

In the preferred embodiment of the invention, the therapeutic powder is located on the aeroelastic element and the aeroelastic vibrations cause the dispersion of the powder as an aerosol. A powder dose applicator is represented in FIG. 5 and dispenses the powder dose to the aeroelastic membrane immediately prior to the dose being inhaled by the patient. The powder dose applicator comprises a dispensing chute (13) filled with at least one dose of powder (14), and a wheel at the bottom end of the dispensing chute turns as the membrane moves beneath the chute. The wheel is notched around its circumference, and the notches fill with powder from the dispensing chute and empty onto the aeroelastic membrane as the wheel turns, resulting in a predetermined dose being applied to the aeroelastic membrane. After the dose falls onto the membrane from the wheel, the membrane passes through two flattening rollers (11), one above and one below the aeroelastic membrane. The rollers turn as the aeroelastic membrane moves from the first spool to the second spool, flattening the powder onto the aeroelastic membrane and breaking up any agglomeration in the powder for optimal dispersal.

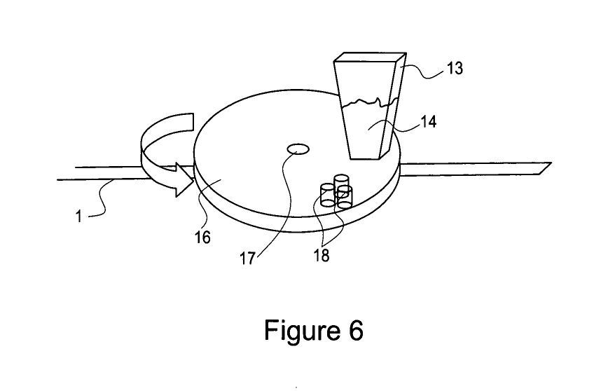

In an alternative embodiment of the invention, the powder dose applicator is the configuration depicted in FIG. 6. The alternate powder dose applicator comprises a dispensing chute (13) above the aeroelastic membrane without a notched wheel for dispensing the proper dose. Instead, a dispensing disk (16) located between the aeroelastic membrane and the dispensing chute, which is in contact with the bottom end of dispensing chute, rotates around its hub (17) as the advancing means is activated. The dispensing disk further comprises multiple dispensing openings (18) clustered in one section of the dispensing disk, resulting in an accurate amount of powder falling through the dispensing openings as the disk rotates past the dispensing chute.

In another embodiment, the aeroelastic element is part of the powder packaging. At least one powder dose is pre-metered into a strip comprising the aeroelastic element and a peelable sealing strip that encapsulates the powder in discrete doses. The sealing strip is removed prior to inhalation by an opening means, exposing the powder to the airflow through the device. The opening means is located where the powder dose applicator is located in the preferred embodiment.

In an alternate embodiment of the invention, the powder dose is pre-metered into blister-strip packaging with a peelable layer protecting each dose until it is ready to be dispensed. The blister strip packaging is coiled onto the first and second rollers, in place of the aeroelastic element. The advancement means advances the blister strip by one dose, and an opening means replaces the powder dose applicator of the preferred embodiment. The opening means strips the peelable layer from the blister strip when the advancing means is activated, exposing a single powder dose for dispensing. In a blister strip embodiment, the aeroelastic element extends across the center dispensing region parallel to the blister strip packaging. The aeroelastic element is held at a pre-determined level of tension by the tensioner rods. The tensioner rods are not attached to the crank or, therefore, the advancing means in this embodiment.

In an alternate embodiment of the invention, the inhaler comprises a single dose of therapeutic powder.

In an alternate embodiment of the invention, the therapeutic powder is in a reservoir or resonance cavity that undergoes aeroelastic vibrations. Additionally, alternative structures may also be used to enhance the dispersal of the powder as long as they show aeroelasticity, such as reeds, sheets, panels and blades. The aeroelastic element may be constructed of materials that show elasticity, comprising polymers, metals, and metal-coated polymers.

The route of the air flowing through the inhaler is illustrated by the arrows (4) in FIG. 3 and is as follows: as the patient inhales, air is sucked into the inhaler through multiple airflow inlets (3) at the back of the inhaler, which extend from the outer surface of the casing into the back end of the open center chamber and over the airflow modifiers (2), which extend from the left wall of the chamber to the right wall; the air engages the aeroelastic membrane (1) which is stretched across the center dispensing region of the chamber, causing the membrane to vibrate or flutter and dispersing the powder dose from the membrane into the airflow; the air and powder are sucked into the inner end of the turbulent airflow nozzle, a cylindrical unit in which at least one tube extends in a helical or coiled fashion from the front end of the center chamber through the outer surface of the casing and into the mouthpiece; the mouthpiece is affixed to the outer surface of the casing and comprises a cylindrical opening that engages the outer end of the nozzle and has a shape that is appropriate for the patient's lips to purse over it and form a seal between the lips and the mouthpiece. The air and powder leave the mouthpiece and enter the patient's mouth and respiratory tract. Both the airflow modifiers and the helical shape of the nozzle increase the turbulence of the airflow and fully aerosolize and break up the powder dose, maximizing the dose received by the patient, and allowing the small particles to pass further into the respiratory tract.

The method for dispensing a powder dose using the dry powder inhaler of the present invention comprises three steps. First, the patient activates the advancement means, which results in a single powder dose being moved into the center dispensing region. Second, the patient purses his or her lips around the mouthpiece, creating a seal. Finally, the patient inhales, resulting in the powder dose being delivered into the patient's respiratory system.

What is claimed is:

1. A dry powder inhaler apparatus for delivering pharmaceuticals to a patient, comprising:
a. a casing, comprising an outer surface and two inner walls, the inner walls forming an open center chamber through which air can flow within the casing and two enclosed chambers, a right chamber located to the right of the open center chamber and a left chamber located to the left of the open center chamber, the center chamber, further comprising a front end, a back end, a left wall, a right wall and a center dispensing region, the back end of the center chamber further comprising airflow modifiers anterior to the airflow inlets, the airflow modifiers further comprising multiple triangular rods extending from the left wall to the right wall, resulting in vortices which allow air to pass above and below the triangular rods
b. multiple airflow inlets extending from the back of the center chamber through the outer surface of the casing;
c. an aeroelastic element spanning the center dispensing region, comprising:
i. an aeroelastic membrane with a powder dose, the membrane having a used end and an unused end, the membrane being wound between two spools, a first spool and a second spool, the first spool comprising the unused end being located in the left chamber and the second spool attaching to the used end being located in the right chamber, resulting in the aeroelastic membrane running through the slot in the left wall across the center dispensing region and through the slot in the right wall onto the second spool;
ii. two axles running through the center of the spools, the axle for the second spool comprising a concentric spring, resulting in the aeroelastic membrane being transferred from the first spool to the second spool as the spring-loaded axle is activated; and
iii. a roller engaging the aeroelastic membrane immediately adjacent to the first spool, resulting in additional tension in the aeroelastic membrane;
d. two base clamps fixedly attached to the floor of the chambers, one in the right chamber and one in the left chamber, upon which the aeroelastic element rests, the clamps being located between the spools and the left and right walls;
e. two upper clamps located above the base clamps, the upper clamps descending atop the base clamps to hold the aeroelastic element in place across the center dispensing region;
f. a crank movably attached to the two upper clamps, said crank causing the upper clamps to raise when the advancing means is activated and the crank moves;
g. two tensioner rods located between the upper clamps and the left and right walls and movably attached to a crank, the tensioner rods descending to a pre-determined level to further tension the aeroelastic element, releasing when the advancing means is activated and the crank moves;
h. a powder dose applicator located between the first spool and the base clamp in the left chamber, the powder dose applicator comprising:
i. a dispensing chute filled with at least one dose of powder, the dispensing chute comprising a top end and a bottom end;
ii. a wheel at the bottom end of the dispensing chute, the wheel comprising a multitude of notches around its circumference, the notches filling with powder from the dispensing chute and emptying onto the aeroelastic membrane as the wheel turns; and
iii. two flattening rollers, one above and one below the aeroelastic membrane between the dispensing chute and the base clamp, the rollers turning as the aeroelastic membrane moves from the first spool to the second spool, fully engaging the powder with the aeroelastic membrane and breaking up clumps in the powder;
i. a turbulent airflow nozzle, comprising an inner end and an outer end, the inner end engaging the front of the chamber, extending through the outer surface of the casing and the outer end extending through the mouth piece, the nozzle further comprising at least one tube through which air and powder can be inhaled, extending from the inner end to the outer end in a helical fashion, thereby increasing the turbulence in the air that flows through the nozzle;
j. a mouthpiece affixed to the outer wall of the casing, comprising a cylindrical opening that engages the outer end of the nozzle, allowing the air and powder to leave the dry powder inhaler and enter the patient's respiratory system upon inhalation, and having an appropriate shape for pursing the patient's lips over the mouthpiece and creating a seal between the lips and the mouthpiece;
k. a dose counter on the outer wall engaged to an advancement means in such a way that the dose counter changes numbers by one when the advancement means is activated; and
l. an advancement means capable of turning the crank to release the upper clamps and tensioner rods, advancing the dose counter, turning the wheel in the dispensing chute, advancing the spring-loaded axle in the second spool by one position to advance the aeroelastic element a predetermined distance from the first spool to the second spool, and moving a powder dose into the center dispensing region.

2. A dry powder inhaler apparatus for delivering pharmaceuticals to a patient according to claim 1, wherein the aeroelastic element is selected from a group comprising: a membrane, film, reed, sheet, panel or blade.

3. A dry powder inhaler apparatus for delivering pharmaceuticals to a patient according to claim 1, wherein the material from which the aeroelastic element is manufactured is selected from a group, comprising polymers, thin metals, and metal-coated polymers.

4. A dry powder inhaler apparatus for delivering pharmaceuticals to a patient according to claim 1, wherein tension controllers are attached to the spool axle, allowing the tension of the aeroelastic membrane to be manually fixed and maintained across the spool axles, obviating the need for tension rods.

5. A dry powder inhaler apparatus for delivering pharmaceuticals to a patient according to claim 1, wherein the powder dose applicator comprises:
a. a dispensing chute above the aeroelastic membrane filled with at least one powder dose; and
b. a dispensing disk that rotates as the advancing means is activated, located between the aeroelastic membrane and the dispensing chute and being in contact with the bottom end of dispensing chute, the dispensing disk further comprising multiple dispensing openings clustered in one section of the dispensing disk, resulting in an accurate amount of powder falling through the dispensing openings as the disk rotates past the dispensing chute.

6. A dry powder inhaler apparatus for delivering pharmaceuticals to a patient according to claim 1, further comprising:
a. blister strip packaging attached to the two spools in place of the aeroelastic element of claim 1, the blister strip packaging comprising at least one individual dosing cup, each filled with a powder dose and covered by a peelable top layer, the dosing cups arranged serially along the length of the strip;
b. an aeroelastic element fixedly stretched across the center dispensing region, the aeroelastic element being parallel to the blister strip packaging in the center dispensing region in such proximity that the aeroelastic element acts as an actuator, making contact with the blister packaging and dispersing the powder dose when the aeroelastic element begins to vibrate during inhalation by the patient; and
c. a powder dose opener replacing the a powder dose applicator, the powder dose opener being capable of removing the top layer from the blister strip packaging for one dose when the blister strip is advanced between the first and second spools;

7. A dry powder inhaler apparatus for delivering pharmaceuticals to a patient according to claim 3, wherein the blister strip packaging comprises clusters pf multiple small dosing cups for simultaneous multiple drug dosing, the clusters arranged serially along the length of the blister strip.

8. A dry powder inhaler apparatus for delivering pharmaceuticals to a patient according to claim 1, wherein the aeroelastic element is an aeroelastic and deformable membrane, comprising at least one individual dosing cup filled with a powder dose in the same form as blister strip packaging, wherein the dosing cup deforms and raises the powder dose to the level of the surrounding membrane.

9. A dry powder inhaler apparatus for delivering pharmaceuticals to a patient according to claim 1, wherein the inhaler comprises a single powder dose.

10. A method for dispensing powder by inhalation using the dry powder inhaler apparatus claimed in claim 1, comprising the steps of:
a. activating the advancement means;
b. having the patient purse the patient's lips around the mouthpiece; and
c. having the patient inhale so the dry powder inhaler delivers a dose of the powder into the patient's respiratory system.
